(19) United States
(12) Patent Application Publication
(10) Pub. No.: US 2025/0151987 A1
(43) Pub. Date: May 15, 2025

(54) ENDOSCOPE TREATMENT TOOL AND ENDOSCOPE SYSTEM

(71) Applicants: Naohisa YAHAGI, Tokyo (JP); OLYMPUS CORPORATION, Tokyo (JP)

(72) Inventors: Naohisa YAHAGI, Tokyo (JP); Junji SHIONO, Yokohama (JP); Yuya MATSUOKA, Tokyo (JP); Yuya HIDAKA, Aomori (JP); Yasuhiro TABUCHI, Aomori (JP)

(73) Assignees: Naohisa YAHAGI, Tokyo (JP); OLYMPUS CORPORATION, Tokyo (JP)

(21) Appl. No.: 19/024,579

(22) Filed: Jan. 16, 2025

Related U.S. Application Data
(63) Continuation of application No. 17/377,533, filed on Jul. 16, 2021.
Application No. 17/377,533 is a continuation-in-part of application No. 16/990,088, filed on Aug. 11, 2020, now Pat. No. 11,730,343.
Application No. 16/990,088 is a continuation of application No. PCT/JP2018/006677, filed on Feb. 23, 2018.

(51) Int. Cl. A61B 1/00; A61B 17/00; A61B 17/29

(52) U.S. Cl. CPC A61B 1/0014; A61B 1/00137; A61B 17/00234; A61B 17/29; A61B 2017/00296

(57) ABSTRACT

An endoscope treatment tool includes: a cap defining a longitudinal axis and a proximal-end opening for insertion of a distal end section of an endoscope to detachably attach the cap to the endoscope; a treatment tool extending longitudinally along an outside of the cap and having a distal end configured for tissue manipulation; and a coupling member extending between the cap and the treatment tool to movably couple the treatment tool to the cap, the coupling member having high flexibility.

### CROSS-REFERENCE TO RELATED APPLICATIONS

This is a continuation of U.S. patent application Ser. No. 17/377,533, filed on Jul. 16, 2021, which is a continuation-in-part of U.S. application Ser. No. 16/990,088, filed on Aug. 11, 2020, which are each hereby incorporated herein by reference in their entirety.

### TECHNICAL FIELD

The present invention relates to an endoscope treatment tool and an endoscope system.

### BACKGROUND ART

There is a known connecting tool that attaches a guide tube for guiding a treatment tool to a distal end of an insertion portion of an endoscope (for example, see PTL 1).

According to this connecting tool, the distal end of the treatment tool guided through the guide tube, which is disposed radially outside the insertion portion of the endoscope, is disposed inside the field of view of the endoscope, and treatment can be performed on an affected area while observing the distal end of the treatment tool.

### CITATION LIST

### Patent Literature

{PTL 1}Japanese Unexamined Patent Application, Publication No. 2005-334237

### SUMMARY OF INVENTION

One aspect of the present invention is directed to an endoscope treatment tool including: a cap defining a longitudinal axis and a proximal-end opening for insertion of a distal end section of an endoscope to detachably attach the cap to the endoscope; a treatment tool extending longitudinally along an outside of the cap and having a distal end configured for tissue manipulation; and a coupling member extending between the cap and the treatment tool to movably couple the treatment tool to the cap, the coupling member having high flexibility.

### BRIEF DESCRIPTION OF DRAWINGS

FIG. 1A is a side view showing the configuration of an endoscope treatment tool and an endoscope system according to one embodiment of the present invention.

FIG. 1B is a front view of the endoscope treatment tool shown in FIG. 1A, viewed from a distal end thereof in the longitudinal direction.

FIG. 2 is a side view of the endoscope treatment tool shown in FIG. 1A, for explaining the operation of a coupling member and a grasping part.

FIG. 3 is a view for explaining how to use the endoscope system shown in FIG. 1A.

FIG. 4 is a side view of a modification of the endoscope treatment tool shown in FIG. 1A.

FIG. 5 is a side view of another modification of the endoscope treatment tool shown in FIG. 1A.

FIG. 6 is a side view of still another modification of the endoscope treatment tool shown in FIG. 1A.

FIG. 7 is a partially enlarged view for explaining attachment of the coupling member to the treatment tool, in still another modification of the endoscope treatment tool shown in FIG. 1A.

FIG. 8 is a front view showing a rotating member provided in the treatment tool shown in FIG. 6, with a section of the rotating member being shown in cross section.

FIG. 9 is a side view showing still another modification of the endoscope treatment tool shown in FIG. 1A.

FIG. 10 is a side view showing a state in which affected tissue is raised by a large amount by an endoscope treatment tool shown in FIG. 9.

FIG. 11 is a side view showing still another modification of the endoscope treatment tool shown in FIG. 1A.

FIG. 12 is a side view showing still another modification of the endoscope treatment tool shown in FIG. 1A.

FIG. 13 is a partially enlarged sectional view for explaining fixing of the coupling member to a cap, in an endoscope treatment tool shown in FIG. 12.

FIG. 14 is a perspective view partially showing the cap of the endoscope treatment tool shown in FIG. 12.

FIG. 15 is a partial side view for explaining the shape of a distal-end edge of the cap shown in FIG. 14.

FIG. 16 is a view showing the overall configuration of another example of the endoscope treatment tool shown in FIG. 1A.

FIG. 17 is a partial longitudinal sectional view for explaining attachment of the treatment tool to the cap, in an endoscope treatment tool shown in FIG. 16.

FIG. 18 is a side view showing the cap of the endoscope treatment tool shown in FIG. 16.

FIG. 19 is a view for explaining a fixture for fixing the cap of the endoscope treatment tool shown in FIG. 16 to an insertion portion of an endoscope.

FIG. 20 is a transverse sectional view of the cap to which the fixture shown in FIG. 19 is attached.

FIG. 21 is a transverse sectional view showing a state in which the cap has been fixed to the insertion portion by using the fixture shown in FIG. 20.

FIG. 22 is a partial longitudinal sectional view showing a grip part of the endoscope treatment tool shown in FIG. 16.

FIG. 23 is a longitudinal sectional view of a connector of the endoscope treatment tool shown in FIG. 16.

FIG. 24 is a longitudinal sectional view showing a state in which a lock button is not pressed, in the grip part shown in FIG. 22.

FIG. 25 is a longitudinal sectional view showing a state in which the lock button is pressed, in the grip part shown in FIG. 22.

FIG. 26 is a side view for explaining an operation of advancing grasping forceps, in the grip part shown in FIG. 25.

FIG. 27 is a side view for explaining an operation of retracting the grasping forceps, in the grip part shown in FIG. 25.

FIG. 28 is a partial longitudinal sectional view for explaining the position of a metal part in the grip part shown in FIG. 27.

FIG. 29 is a partial longitudinal sectional view for explaining the position of the metal part in the grip part shown in FIG. 26.

FIG. 30 is a side view for explaining an operation for opening the grasping part, in the endoscope treatment tool shown in FIG. 16.

FIG. 31 is a side view for explaining an operation for closing the grasping part, in the endoscope treatment tool shown in FIG. 16.

FIG. 32 is a side view for explaining an operation for locking the grasping part in a closed state, in the endoscope treatment tool shown in FIG. 16.

FIG. 33 is a perspective view showing an intermediate fixing part in the endoscope treatment tool shown in FIG. 16.

FIG. 34 is a transverse sectional view showing a state in which the intermediate fixing part shown in FIG. 33 has been fixed to the insertion portion of the endoscope.

### DESCRIPTION OF EMBODIMENTS

An endoscope treatment tool 1 and an endoscope system 100 including the same according to one embodiment of the present invention will be described below with reference to the drawings.

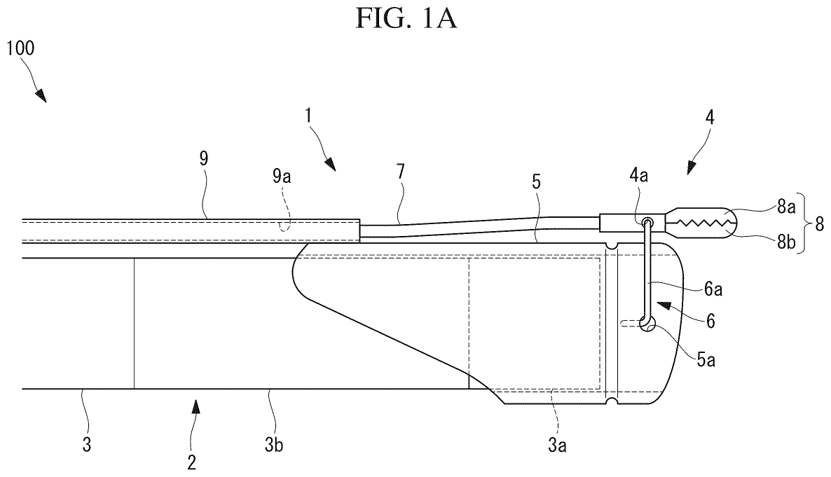

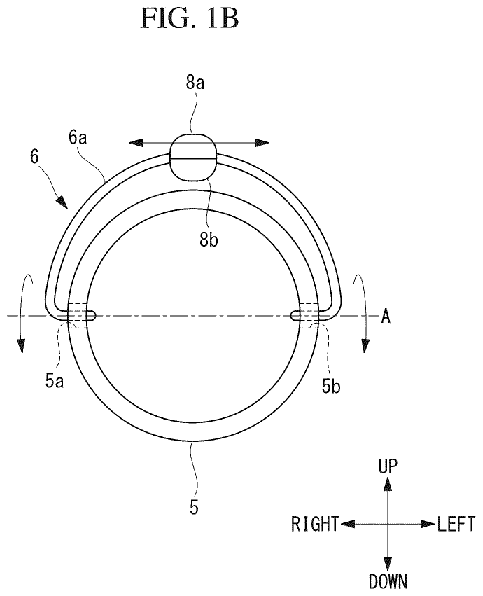

As shown in FIGS. 1A and 1B, the endoscope system 100 of this embodiment includes: an endoscope 2 that has a long insertion portion 3 to be inserted into a body; and an endoscope treatment tool 1 that can be detachably attached to a distal-end section of the insertion portion 3.

The insertion portion 3 has: a hard distal-end rigid section 3a that includes a distal-end surface of the insertion portion 3 and that incorporates a lens etc.; and a bending section 3b that is adjacent to a proximal end of the distal-end rigid section 3a and that can be bent. The insertion portion 3 has an up-down direction and a left-right direction that are each perpendicular to the longitudinal axis and that are perpendicular to each other. The up-down direction and the left-right direction of the insertion portion 3 correspond to the up-down direction and the left-right direction of an endoscopic image. The bending section 3b can be bent at least in the left-right direction.

The endoscope treatment tool 1 includes: a treatment tool 4, such as grasping forceps; a substantially cylindrical cap 5 that can be detachably attached to an outer side of the distal-end section of the insertion portion 3 and that supports the treatment tool 4; and a linear coupling member 6 that couples the treatment tool 4 with the cap 5.

The treatment tool 4 includes a long body part 7 and a grasping part 8 that is connected to a distal end of the body part 7, and is disposed at a radially outer side of the cap 5 along the longitudinal direction of the cap 5.

The body part 7 has flexibility at least at a distal-end section thereof disposed closer to the distal end than a support part 9, to be described later, is.

The grasping part 8 has a pair of grasping pieces 8a and 8b that can open and close with respect to each other, thus making it possible to grasp living tissue between the pair of grasping pieces 8a and 8b.

The cap 5 is made of an elastic material, such as elastomer, and is open at a distal-end surface thereof and a proximal-end surface thereof. The distal-end section of the insertion portion 3 is inserted into the cap 5 from the opening at the proximal-end surface of the cap 5 and is fitted into the cap 5, thereby making it possible to attach the cap 5 to the insertion portion 3. The length of the cap 5 is longer than the length of the distal-end rigid section 3a, so that, in a state in which the cap 5 is attached to the distal-end section of the insertion portion 3, a distal-end section and a proximal-end section of the cap 5 respectively protrude from a distal end and a proximal end of the distal-end rigid section 3a, in the longitudinal direction.

As shown in FIG. 1B, the cap 5 has the up-down direction (first radial direction) and the left-right direction (second radial direction) that are each perpendicular to the longitudinal axis and are perpendicular to each other. As shown in FIG. 1A, the support part 9, which supports the body part 7 of the treatment tool 4 so as to be movable in the longitudinal direction, is provided at an upper side of the proximal-end section of the cap 5. The support part 9 is formed of, for example, a tube whose distal-end section is fixed to an outer circumferential surface of the proximal-end section of the cap 5 and that extends toward the opposite side from the distal end of the cap 5 along the longitudinal direction of the cap 5, and has a channel 9a that penetrates through the support part 9 in the longitudinal direction and that has an inner diameter greater than an outer diameter of the body part 7. The body part 7 is inserted through the channel 9a, is disposed along the longitudinal direction of the cap 5, and can be moved inside the channel 9a in the longitudinal direction. In this way, the support part 9 is provided on the proximal-end section of the cap 5, thereby making it possible to secure a sufficient length of a section of the body part 7 that protrudes from the support part 9 toward the distal end.

The coupling member 6 is a small-diameter member made of a material that hardly or never expands or contracts in the longitudinal direction and that has flexibility, and generates tension when a tensile force acts thereon in the longitudinal direction. For example, a thread, a metal wire, or a rubber is suitably used as the coupling member 6.

The coupling member 6 is inserted through a hole 4a that is provided in the treatment tool 4 and holes 5a and 5b that are provided in the cap 5, thereby coupling the cap 5 with the treatment tool 4.

Specifically, the hole 4a, which penetrates through the treatment tool 4 in a direction perpendicular to the longitudinal direction of the body part 7, is provided in the treatment tool 4 at a position between the body part 7 and the grasping part 8. The hole 4a has an inner diameter greater than an outer diameter of the coupling member 6, thus allowing the coupling member 6 to move inside the hole 4a in the longitudinal direction of the coupling member 6. Preferably, the hole 4a is a long hole.

The two holes 5a and 5b, which each radially penetrate the cap 5 from an outer circumferential surface to an inner circumferential surface of the cap 5, are provided in the distal-end section of the cap 5 at two positions that are spaced in the circumferential direction and that are opposed to each other in the left-right direction. The holes 5a and 5b have inner diameters greater than the outer diameter of the coupling member 6, thus allowing the coupling member 6 to rotate, inside the holes 5a and 5b, about the center axes of the holes 5a and 5b.

The coupling member 6 has a swing section 6a that is disposed outside the distal-end section of the cap 5 and that is inserted through the hole 4a of the treatment tool 4. As shown in FIG. 1B, the swing section 6a is formed of a substantially arc-shaped section having a fixed length. Both end sections of the coupling member 6 that sandwich the swing section 6a in the longitudinal direction are inserted through the holes 5a and 5b from the outside of the cap 5 to the inside thereof and are fixed to an inner surface of the cap 5. Accordingly, the swing section 6a is supported by the cap 5 so as to be swingable about a swing axis A that passes through the two holes 5a and 5b in the left-right direction.

With the treatment tool 4 and the cap 5 being coupled via the swing section 6a, the grasping part 8 can be moved in front of the cap 5 in the up-down direction through a push-pull operation of the treatment tool 4 in the longitudinal direction.

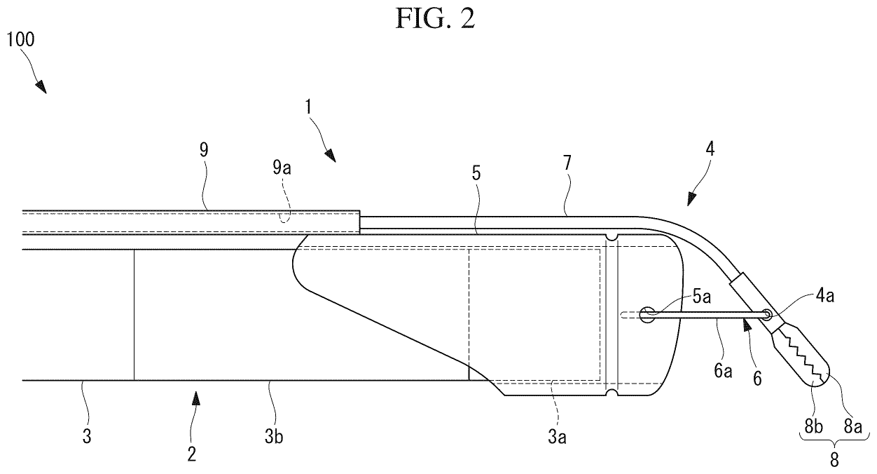

Specifically, as shown in FIG. 2, when the treatment tool 4 is pushed frontward in the longitudinal direction, the swing section 6a of the coupling member 6 pushed by the treatment tool 4 generates a tensile force and swings about the swing axis A from an upper side to a lower side, and the treatment tool 4, which is coupled with the swing section 6a, protrudes frontward while bending the body part 7 downward. Accordingly, the grasping part 8 moves from the upper side toward the lower side, in front of the cap 5. Furthermore, when the treatment tool 4 is pulled rearward in the longitudinal direction, the swing section 6a swings from the lower side to the upper side, and the grasping part 8 moves from the lower side to the upper side, in front of the cap 5.

Here, in a state in which a pushing force or a pulling force is not applied by the treatment tool 4, the swing section 6a is in a relaxed state, thus making it possible to easily move inside the hole 4a. Therefore, when the swing section 6a is in the relaxed state, relative movement of the treatment tool 4 and the cap 5 in the left-right direction is allowed by the flexibility of the swing section 6a and through movement of the swing section 6a inside the hole 4a. It is preferred that the hole 4a extend along the left-right direction of the cap 5, so as to allow more smooth relative movement of the treatment tool 4 and the cap 5 in the left-right direction.

Next, the operation of the thus-configured endoscope treatment tool 1 and endoscope system 100 will be described below.

In order to perform treatment on affected tissue X in the body of a patient by using the endoscope system 100 of this embodiment, as shown in FIG. 1A, the distal-end section of the insertion portion 3 is fitted into the cap 5, thereby attaching the cap 5 and the treatment tool 4 to the distal-end section of the insertion portion 3. The cap 5 and the insertion portion 3 are fixed to each other by the friction between the inner circumferential surface of the cap 5 and an outer circumferential surface of the insertion portion 3. At this time, the support part 9 and the body part 7 are disposed above the insertion portion 3, and the relative positions of the cap 5 and the insertion portion 3 about the longitudinal axis are adjusted such that the up-down direction and the left-right direction of the cap 5 are respectively aligned with the up-down direction and the left-right direction of the insertion portion 3. Furthermore, as shown in FIGS. 1A and 1B, the grasping part 8 is disposed at a position higher than the cap 5.

Next, the endoscope 2 is actuated, the assembly of the insertion portion 3 and the endoscope treatment tool 1 is inserted into the body while checking an endoscopic image in front of the distal-end surface of the insertion portion 3, and the distal end of the insertion portion 3 is disposed such that the affected tissue X is located in a lower area of the field of view of the endoscope 2. Then, the proximal-end section of the treatment tool 4, which is disposed outside the body, is pushed toward the distal end, to move the grasping part 8 frontward in the longitudinal direction, with respect to the cap 5 and the insertion portion 3.

Through the frontward movement of the grasping part 8, the coupling member 6 swings about the swing axis A, thus bending the body part 7 downward and moving the grasping part 8 from the upper side to the lower side. Therefore, in the endoscopic image, the grasping part 8 appears from the upper side and moves to the lower side toward the affected tissue X. Accordingly, merely by moving the treatment tool 4 frontward and making the grasping part 8 protrude from the distal end of the cap 5, it is possible to make the grasping part 8 reach the affected tissue X.

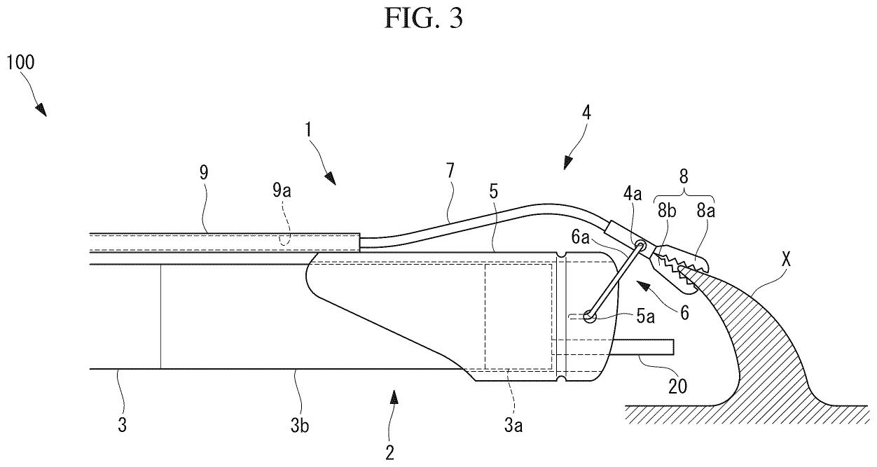

Next, the grasping part 8 that has been made to reach the affected tissue X is operated, the affected tissue X is grasped by the grasping part 8, as shown in FIG. 3, and the proximal-end section of the treatment tool 4 is pulled to move the grasping part 8 toward the proximal end, thereby raising the grasped affected tissue X. In this state, the distance between the raised affected tissue X and the distal-end surface of the insertion portion 3 is secured by the distal-end section of the cap 5, which protrudes from the distal-end surface of the insertion portion 3. Next, the pulling force applied to the proximal-end section of the treatment tool 4 is released to put the swing section 6a in the relaxed state, and a treatment tool 20, such as an electric scalpel, introduced via a channel (not shown) provided in the insertion portion 3 is displaced through a bending operation of the bending section 3b in the left-right direction, thereby cutting off the raised affected tissue X by means of the treatment tool 20.

In this case, according to this embodiment, the coupling member 6, which is inserted through the hole 4a in the treatment tool 4 so as to be movable therein, couples the treatment tool 4 and the cap 5 in a manner allowing relative movement in the left-right direction. Therefore, when the bending section 3b is bent in the left-right direction, the coupling member 6 moves in the left-right direction with respect to the treatment tool 4, thereby preventing the movement of the bending section 3b in the left-right direction from being transferred to the treatment tool 4 via the coupling member 6. Accordingly, the treatment tool 4 is prevented from following the bending section 3b. Specifically, in the left-right direction, movement of the grasping part 8 and movement of the bending section 3b can be independent of each other. Accordingly, there is an advantage in that, regardless of the motion of the bending section 3b in the left-right direction when the affected tissue X is cut off, the grasping part 8 raising the affected tissue X can be kept in a stationary state, and the task of cutting off the affected tissue X can be easily performed in a state in which a tensile force is applied to the affected tissue X.

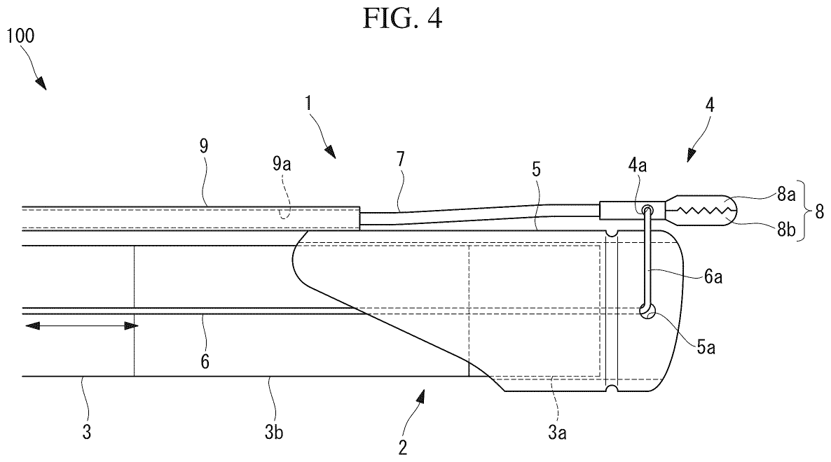

In this embodiment, although the coupling member 6 is inserted through the hole 4a in the treatment tool 4 so as to be movable therein, instead of this, as shown in FIG. 4, the coupling member 6 may be inserted through the holes 5a and 5b in the cap 5 so as to be movable therein in the longitudinal direction of the coupling member 6. The coupling member 6 extends from the holes 5a and 5b toward the proximal end along the longitudinal direction of the insertion portion 3, and both ends of the coupling member 6 are disposed at the proximal end of the insertion portion 3. In this case, an intermediate position of the swing section 6a in the longitudinal direction is fixed to the treatment tool 4.

By doing so, when the bending section 3b is bent in the left-right direction, both end sections of the swing section 6a move inside the holes 5a and 5b, thereby making it possible to allow relative movement of the treatment tool 4 and the cap 5 in the left-right direction.

In a modification shown in FIG. 4, the tensile force on the coupling member 6 is changed through an operation of both ends of the coupling member 6, thereby making it possible to switch the swing section 6a between a tensed state in which a tensile force is applied and a relaxed state. Specifically, when the affected tissue X is grasped by using the grasping part 8, the positions of both ends of the coupling member 6 are fixed with respect to the insertion portion 3, and a tensile force is applied to the swing section 6a of the coupling member 6 by the grasping part 8 that has been pushed forward, thereby making it possible to make the swing section 6a and the grasping part 8 swing about the swing axis A. Furthermore, after the affected tissue X is raised by the grasping part 8, both ends of the coupling member 6 are unfixed, and the length of the swing section 6a is increased a little through an operation of both ends of the coupling member 6, thus making it possible to make the swing section 6a relax. Therefore, after the affected tissue X is raised by the grasping part 8, as shown in FIG. 3, the swing section 6a is released, and the treatment tool 4 is pushed forward in the longitudinal direction, thereby making it also possible to raise the affected tissue X in a direction away from the distal end of the insertion portion 3 in the longitudinal direction.

Furthermore, in the modification shown in FIG. 4, by pushing the insertion portion 3 and the treatment tool 4 forward in the state in which the affected tissue X is raised by the grasping part 8, as shown in FIG. 3, it is possible to push the affected tissue X in a direction away from the distal end of the cap 5 by using the grasping part 8. Furthermore, in this state, the swing section 6a is relaxed through an operation of both ends of the coupling member 6, thereby making it possible to move the treatment tool 4 and the insertion portion 3 independently of each other. Therefore, while pushing the affected tissue X by using the grasping part 8, the affected tissue X can be cut off through a bending operation of the bending section 3b in the left-right direction.

Alternatively, in this embodiment, it is also possible to fix the coupling member 6 to both the treatment tool 4 and the cap 5 and to allow relative movement of the treatment tool 4 and the cap 5 in the left-right direction due to the flexibility of the swing section 6a. In this case, it is preferable that a member having high flexibility, such as a thread, be used as the coupling member 6. It is preferred that a stranded suture made of polyethylene terephthalate be used as a thread.

Furthermore, in the modification shown in FIG. 4, at least an upper section of the cap 5 may be formed of an elastic member that can be bent radially inward.

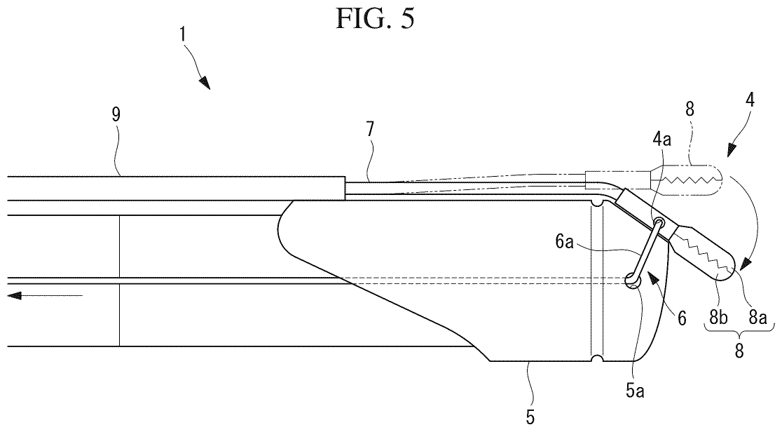

By doing so, as shown in FIG. 5, when both ends of the coupling member 6 are pulled, the grasping part 8 moves downward while bending the upper section of the cap 5 radially inward. Accordingly, the grasping part 8 can be disposed at a position even closer to the distal-end surface of the insertion portion 3.

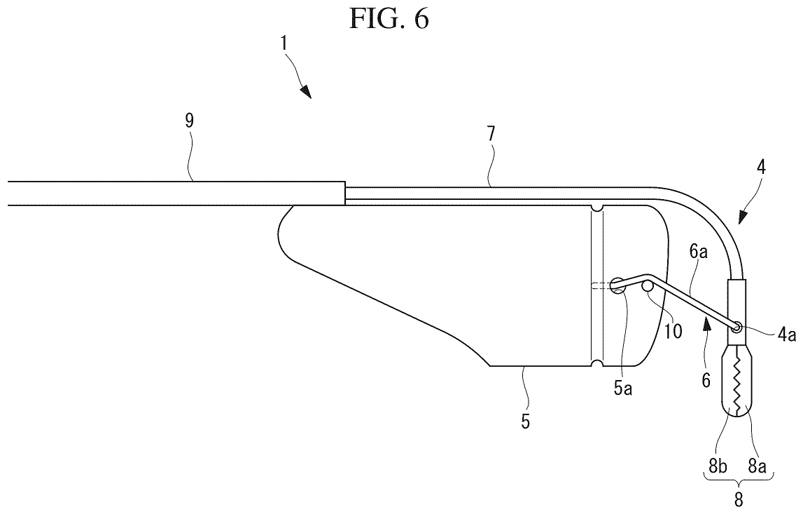

In this embodiment, as shown in FIG. 6, a protrusion 10 that radially protrudes from the outer circumferential surface of the cap 5 may be provided thereon. The protrusion 10 is provided so as to be closer to the distal end than the swing axis A (i.e., the holes 5a and 5b) is, and is preferably provided at a position aligned with or higher than the swing axis A in the up-down direction.

By doing so, when the swing section 6a swings downward, an intermediate position of the swing section 6a is caught by the protrusion 10 and is bent, thereby making it possible to dispose the grasping part 8 at a position even closer to the distal-end surface of the insertion portion 3.

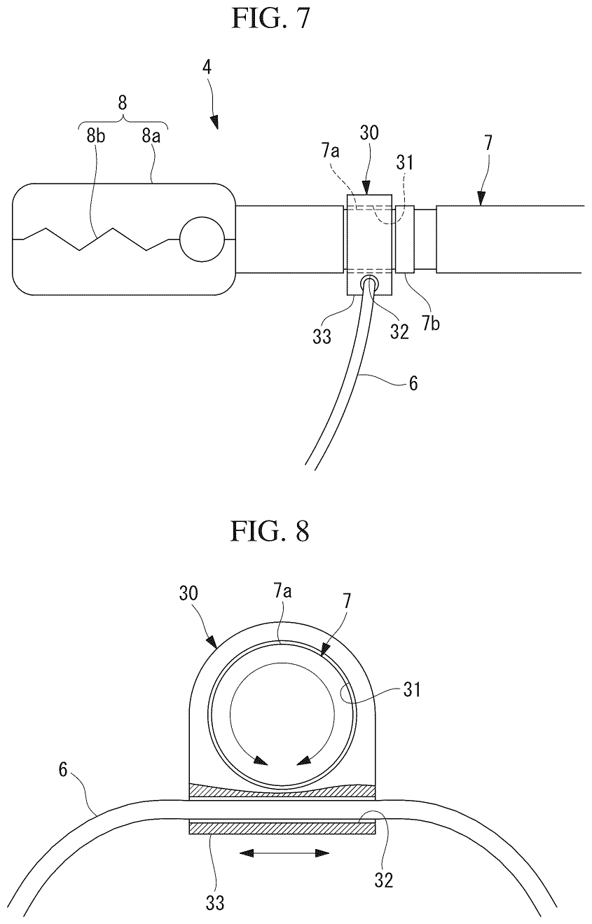

The coupling member 6 is inserted through the hole 4a, which penetrates in a direction perpendicular to the longitudinal direction of the body part 7, so as to be movable in the longitudinal direction of the coupling member 6. Instead of this, as shown in FIGS. 7 and 8, the coupling member 6 may be movably inserted through a second through-hole 32, to be described later, in a cylindrical rotating member 30 through which the body part 7 is inserted.

As shown in FIG. 7, the body part 7 includes, at a region close to the distal end, a recessed section 7a an outer diameter dimension of which is less than an outer diameter dimension of the body part 7 at the other region, and a stopper part 7b that is attached to the recessed section 7a. The stopper part 7b is a ring member that has an outer diameter dimension slightly larger than an inner diameter dimension of a first through-hole 31, to be described later, and can be fixed to the recessed section 7a.

As shown in FIGS. 7 and 8, the rotating member 30 includes the first through-hole 31, which is provided along the axial direction and which has the inner diameter dimension slightly larger than the outer diameter dimension of the recessed section 7a of the body part 7. Furthermore, the rotating member 30 includes the second through-hole 32, which is disposed so as to be in such a positional relationship that the first through-hole 31 and the second through-hole 32 serve as skew lines and which penetrates the rotating member 30 in a direction along an axis perpendicular to the axis of the first through-hole 31. An outer surface of the rotating member 30 on the second through-hole 32 side is formed of a flat surface 33 parallel to the axis of the second through-hole 32. The flat surface 33 is brought into contact with an outer surface of the cap 5, thereby making it possible to stabilize the orientation of the rotating member 30.

It is preferred that the body part 7 be inserted through the first through-hole 31, and the rotating member 30 be disposed at the recessed section 7a of the body part 7, whereby the rotating member 30 is supported so as to be rotatable about the longitudinal axis of the body part 7 with respect to the body part 7. After the rotating member 30 is disposed at the recessed section 7a of the body part 7, the stopper part 7b may be attached at a position of the recessed section 7a close to a proximal end of the rotating member 30, whereby movement of the rotating member 30 in the axial direction with respect to the body part 7 is restricted.

Furthermore, the rotating member 30 may be disposed at such a position that the outer surface thereof that is formed of the flat surface 33 is located closer to the cap 5 than the treatment tool 4 (the recessed section 7a) is, whereby the coupling member 6, which is inserted through the second through-hole 32, is disposed at such a position as to pass between the treatment tool 4 and the cap 5.

With this configuration, because the coupling member 6 is inserted through the second through-hole 32 of the rotating member 30, rotation of the treatment tool 4 about the axis of the second through-hole 32 is allowed, and the coupling member 6 and the rotating member 30 can be relatively moved in the direction along the axis of the second through-hole 32.

In particular, according to the linear-shaped coupling member 6, for example, it is advantageous in a case in which there arises a need to change the field of view by moving the endoscope 2 with living tissue being grasped by the grasping part 8. Specifically, the coupling member 6 is moved inside the second through-hole 32, or the coupling member 6 is deformed, whereby the grasped state of the living tissue can be maintained without causing the grasping part 8 to follow the movement of the endoscope 2.

In this case, the rotating member 30 is rotated about the longitudinal axis of the body part 7, whereby the second through-hole 32 and the coupling member 6, which is inserted through the second through-hole 32, are disposed in such a positional relationship as not to restrain each other. Accordingly, it is possible to suppress an increase in the friction therebetween and to allow free relative movement.

Furthermore, because the rotating member 30 is attached so as to be rotatable about the longitudinal axis of the body part 7, even when the body part 7 is rotated inside the channel 9a of the support part 9, it is possible to prevent the second through-hole 32, through which the coupling member 6 is inserted, from being rotated about the axis of the first through-hole 31. Accordingly, there is an advantage in that an increase in the resistance between the body part 7 and the coupling member 6 is prevented, thereby making it possible to prevent restriction of the movement of the treatment tool 4 with respect to the coupling member 6.

Specifically, in a case in which the hole 4a, through which the coupling member 6 is inserted, is provided in the body part 7, when the treatment tool 4 is rotated about the longitudinal axis, the coupling member 6 is wrapped around the treatment tool 4 in some cases. In contrast to this, because the rotating member 30 is not rotated even when the treatment tool 4 is rotated about the longitudinal axis, it is possible to prevent the coupling member 6 from being wrapped around the treatment tool 4. Specifically, twisting of the coupling member 6 can be prevented without impairing the swivel function of the treatment tool 4.

According to this embodiment, because the second through-hole 32, through which the coupling member 6 is inserted, is disposed between the treatment tool 4 and the cap 5, the body part 7 of the treatment tool 4 can be always disposed closer to a radially outer side of the cap 5 than the coupling member 6, whereby there is an advantage in that it is possible to prevent a situation in which the movement of the treatment tool 4 is disturbed by the coupling member 6.

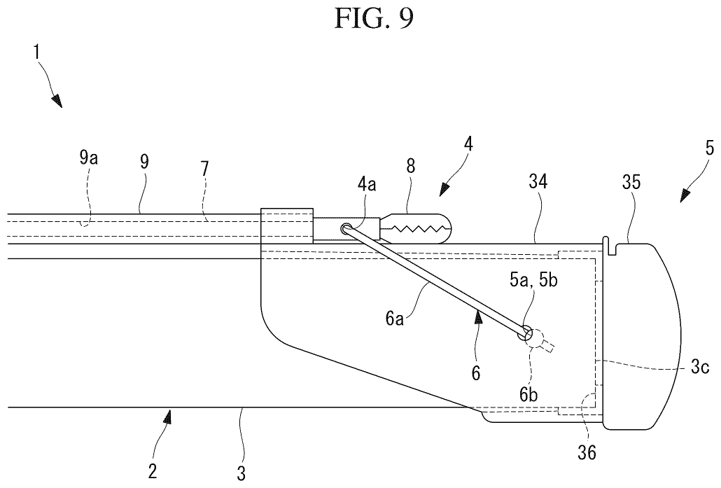

An example case in which the cap 5 is made of an elastic material, such as elastomer, has been illustrated. Instead of this, as shown in FIG. 9, the cap 5 may also include a cylindrical hood part 34 that is made of, for example, polyester elastomer and a cap part 35 that is connected to a distal end of the hood part 34 and that is made of, for example, polycarbonate. The hood part 34 and the cap part 35 may be manufactured through two-color molding.

Since the cap part 35 plays a role in raising surrounding tissue in front of the endoscope 2 in the body and securing a space in front of the endoscope 2, it is preferred that the cap part 35 have such rigidity as to maintain the shape thereof against a force from the surrounding tissue. On the other hand, from the point of view of the ease of attachment to a distal-end section of the endoscope 2, it is preferred that the hood part 34 be formed of an elastic material. By providing an abutment surface 36 on the cap part 35, which has higher rigidity than the hood part 34, the position of a distal-end surface 3c of the insertion portion 3 of the endoscope 2 can be more stabilized.

Both ends of the coupling member 6 are inserted into the two holes 5a and 5b, which are provided in the distal-end section of the cap 5, whereby the coupling member 6 is supported so as to be rotatable about the central axes of the holes 5a and 5b. Instead of this, the coupling member 6 may also be supported by holes 5a and 5b that are provided in the hood part 34, not in the cap part 35 at the distal end. In this case, it is also possible to support the coupling member 6 by inserting both ends of the coupling member 6 into the holes 5a and 5b from the outside toward the inside and forming knots 6b at both ends of the coupling member 6 that are now disposed inside the hood part 34. The knots 6b have widths greater than the inner diameters of the holes 5a and 5b. It is also possible to rotatably support the coupling member 6 by inserting both ends of the coupling member 6 into the holes 5a and 5b from the outside toward the inside and bonding the ends to recessed sections 38.

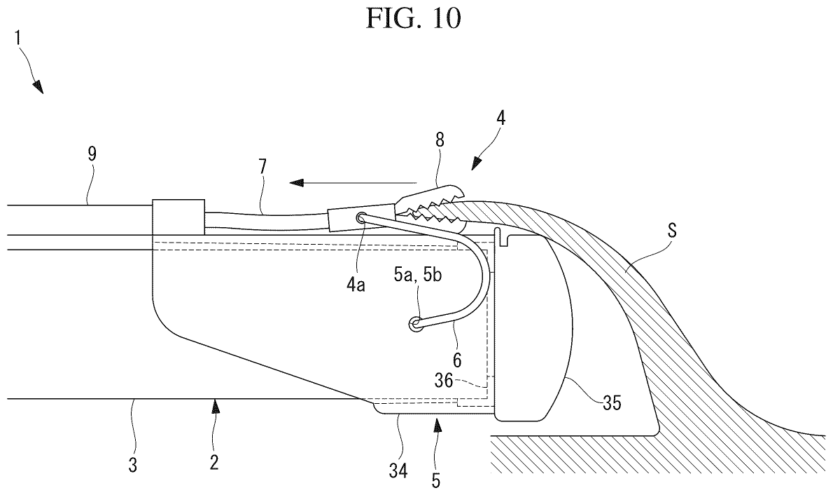

With the holes 5a and 5b being provided in the hood part 34, the grasping part 8 can be disposed at a position closer to the proximal end, compared with a case in which the holes 5a and 5b are provided in the cap part 35. Accordingly, in a state in which the grasping part 8 is fully retracted, the grasping part 8 can be disposed at such a position as not to interfere with surrounding tissue around the distal end of the cap 5. Furthermore, as shown in FIG. 10, it is possible to secure a certain retraction amount of affected tissue S grasped and raised by the grasping part 8 and to expand the raised range of the affected tissue S.

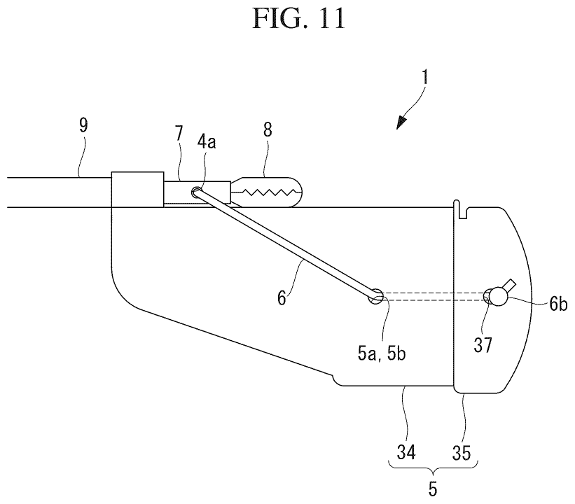

Alternatively, as shown in FIG. 11, the cap part 35 may also have a pair of fixing holes 37 used to fix both end sections of the coupling member 6.

The pair of fixing holes 37 penetrate the cap part 35 in radial directions from an outer circumferential surface thereof to an inner circumferential surface thereof.

The end sections of the coupling member 6 are inserted through the fixing holes 37 from the inside to the outside and are fixed at the fixing holes 37 by the knots 6b formed at the ends of the coupling member 6. Therefore, as shown in FIG. 11, in a case in which the single fixing hole 37 is provided for each of the end sections of the coupling member 6, the knot 6b is disposed outside the cap part 35.

In a case in which the end sections of the coupling member 6 are fixed at the holes 5a and 5b that are provided in the hood part 34, forces tend to be applied to the end sections of the coupling member 6 when the coupling member 6 swings. By fixing the end sections of the coupling member 6 at the fixing holes 37, which are different from the holes 5a and 5b, it is possible to prevent forces from being applied to the end sections when the coupling member 6 swings, and to prevent the end sections of the coupling member 6 from coming off from the cap part 35.

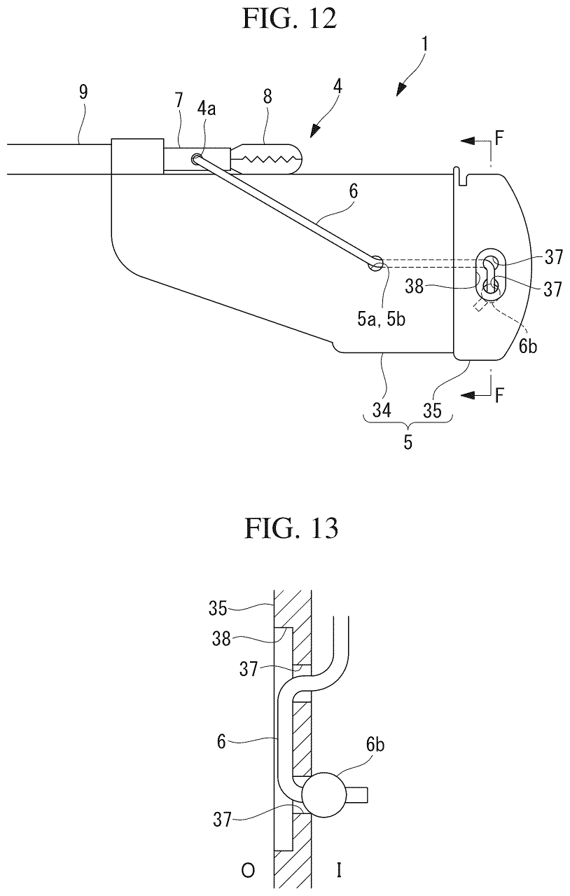

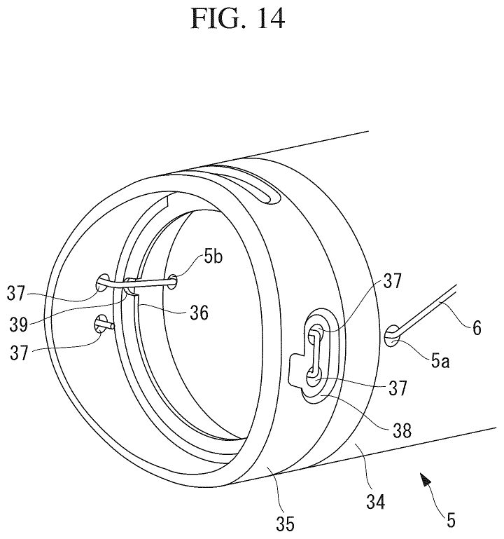

Furthermore, as shown in FIGS. 12 and 13, the recessed sections 38 may be formed around the fixing holes 37. The recessed sections 38 are formed on the outer circumferential surface of the cap part 35 and are recessed radially inward. By providing the recessed sections 38, the coupling member 6 can be disposed within the outer diameter of the cap part 35, as shown in FIG. 13, whereby it is possible to prevent the coupling member 6 from being in contact with the affected tissue S. Adhesive agents may be filled inside the recessed sections 38. In FIG. 13, the left side corresponds to an outer side O of the cap part 35, and the right side corresponds to an inner side I of the cap part 35. Note that, in a case in which the coupling member 6 is fixed to the cap part 35 by the adhesive agents, the knots 6b may be omitted.

The coupling member 6, which is inserted through the holes 5a and 5b of the hood part 34 from the outside to the inside, passes inside the cap 5 and is then inserted through the fixing holes 37, which are provided in the cap part 35, from the inside to the outside. In this case, the coupling member 6 needs to pass inside the cap 5 from the hood part 34 to the cap part 35. Thus, as shown in FIG. 14, notches 39 through which the coupling member 6 is made to pass may also be provided in the abutment surface 36, against which the distal-end surface 3c of the insertion portion 3 of the endoscope 2 is made to abut.

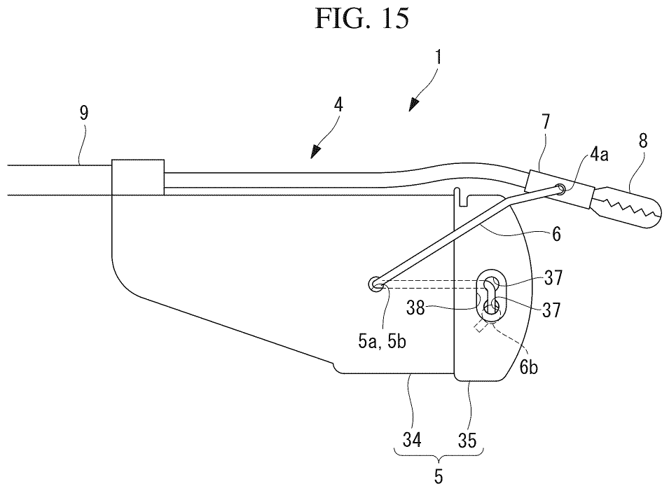

As shown in FIGS. 14 and 15, it is preferred that a distal-end edge of the cap part 35 have a curved shape that smoothly curves so as to protrude farthest forward in the left and right sections thereof and so as to be recessed most in the upper and lower sections thereof. In an operation for advancing the grasping part 8, as shown in FIG. 15, when tension is generated in the coupling member 6, the coupling member 6, which connects the left and right outer surfaces of the hood part 34 and the proximal end of the grasping part 8, is bent at the distal-end edge of the cap part 35.

With the distal-end edge of the cap part 35 being recessed in the upper section thereof, it is possible to delay the start of bending of the coupling member 6 during the forward movement of the grasping part 8 and to suppress the bending angle thereof. As a result, it is possible to reduce the friction resistance between the cap part 35 and the coupling member 6 and to smoothly lower the grasping part 8. In particular, the cap part 35 is made of a rigid material, whereby it is possible to prevent the coupling member 6 from biting into the distal-end edge of the cap part 35 and to further reduce the friction resistance.

Furthermore, with the distal-end edge of the cap part 35 being recessed in the lower section thereof, there is an advantage in that the cap part 35 can be easily slipped under the raised affected tissue S.

Next, another example of the endoscope treatment tool will be illustrated below.

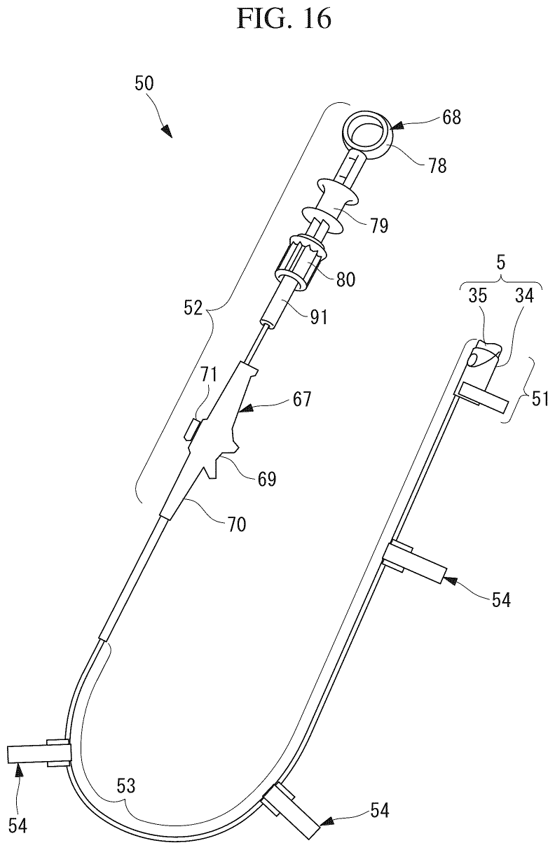

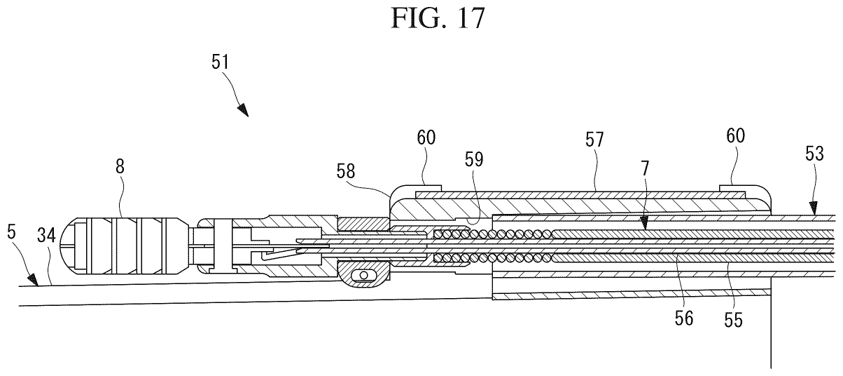

As shown in FIG. 16, an endoscope treatment tool 50 includes a distal-end portion 51, a hand operation portion 52, a channel tube 53 that couples the distal-end portion 51 and the hand operation portion 52, and one or more (for example, three) intermediate fixing parts 54 that are arranged at intervals in the longitudinal direction of the channel tube 53. As shown in FIG. 17, the body part 7, which connects the grasping part 8 in the distal-end portion 51 with the hand operation portion 52, is inserted into the channel tube 53 so as to be movable in the longitudinal direction of the channel tube 53. The body part 7 includes, for example, a coil tube 55 and a wire 56 that is accommodated inside the coil tube 55 so as to be movable in the longitudinal direction.

The distal-end portion 51 includes the grasping part 8, the cap 5, the coupling member 6, and a fixture 57 that fixes the cap 5 to the insertion portion 3 of the endoscope 2.

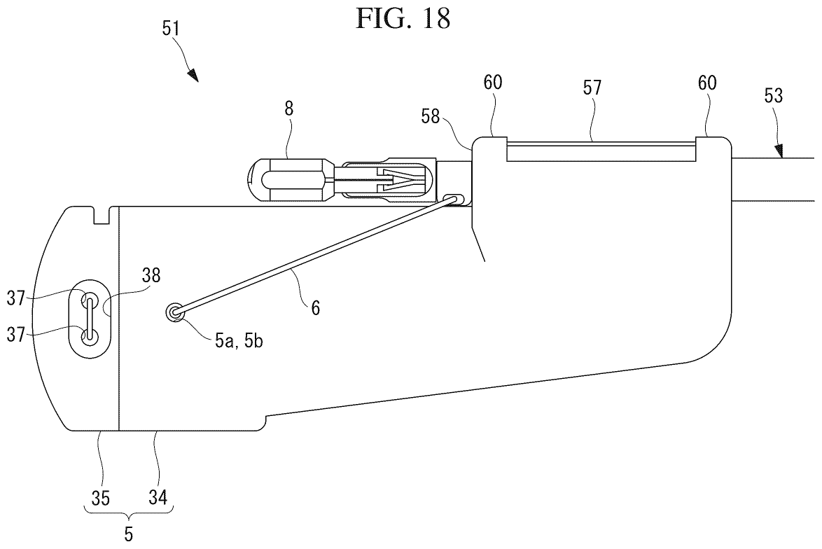

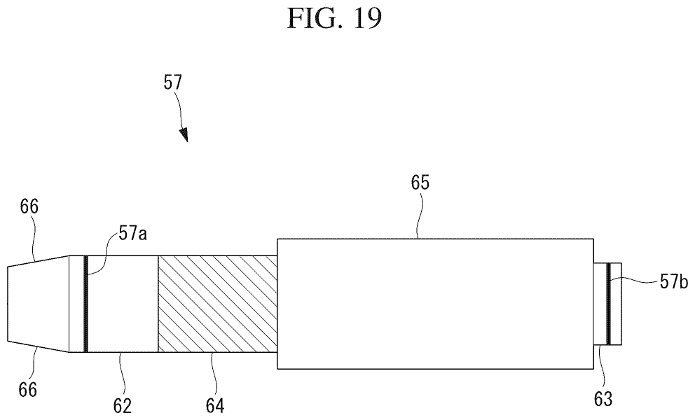

The cap 5 includes the rigid cap part 35 and the flexible hood part 34, as in FIG. 12. As shown in FIG. 18, a protruding section 58 is provided on an upper side of a proximal-end section of the hood part 34, the protruding section 58 being formed integrally with the hood part 34. A tunnel 59 that penetrates the protruding section 58 in the longitudinal direction of the hood part 34 is formed in the protruding section 58, and a distal end of the channel tube 53 is fixed to the protruding section 58 in a state of being inserted into the tunnel 59.

A pair of protrusions 60 that protrude with a gap therebetween in the front-back direction, the gap being slightly larger than the width dimension of one end of the fixture 57, are provided at the apex of the protruding section 58 so as to become indicators used when the fixture 57 is applied.

At an opening of a proximal-end surface of the hood part 34, which is formed of an elastic material, a notch 61 that is obtained by cutting off a circumferential section (lower side) of the hood part 34 up to an intermediate position in the longitudinal direction is provided, whereby a section of the outer circumferential surface of the insertion portion 3 of the endoscope 2, which is inserted into the cap 5, is exposed from the notch 61.

As shown in FIG. 19, the fixture 57 is a tape that includes, on a back surface thereof, a first adhesive surface 62 disposed at one end of the fixture 57 in the longitudinal direction, a second adhesive surface 63 disposed at the other end thereof, and a non-adhesive area 64 disposed at the center thereof. Release paper 65 is applied to the second adhesive surface 63. On a front surface of the fixture 57, straight colored lines 57a and 57b that extend in the width direction are drawn in the vicinities of both ends. Notches 66 that are made to abut against the protrusions 60 are provided at the one end of the fixture 57 at both sides in the width direction.

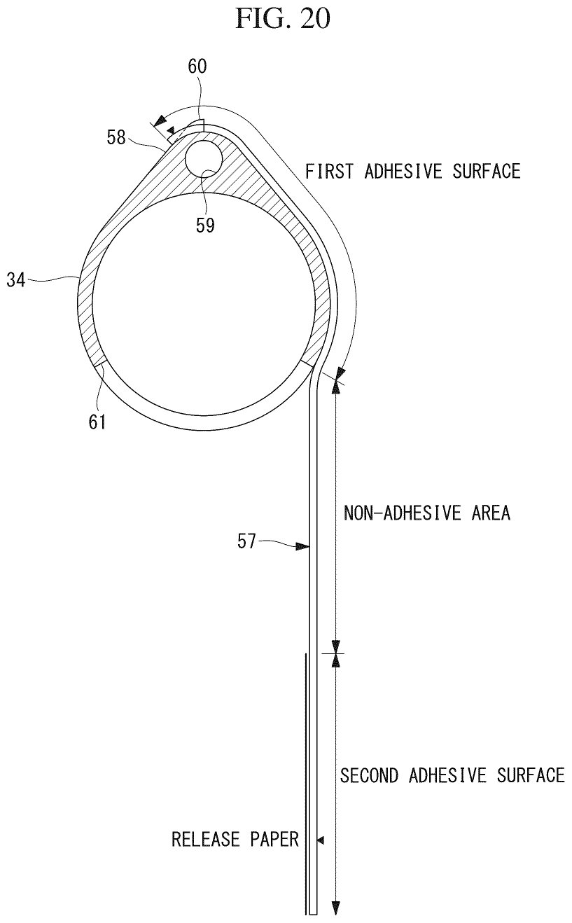

As shown in FIG. 20, the one end of the fixture 57 where the first adhesive surface 62 is provided is disposed between the protrusions 60 on the protruding section 58 of the hood part 34, and the notches 66 of the fixture 57 are made to abut against the protrusions 60, whereby the fixture 57 is positioned in the circumferential direction and the longitudinal direction of the hood part 34. Accordingly, the first adhesive surface 62 is adhered to the outer circumferential surface of the hood part 34 in a state in which the colored line 57a at the one end of the fixture 57 extends in the longitudinal direction of the hood part 34 in the vicinity of the apex of the protruding section 58.

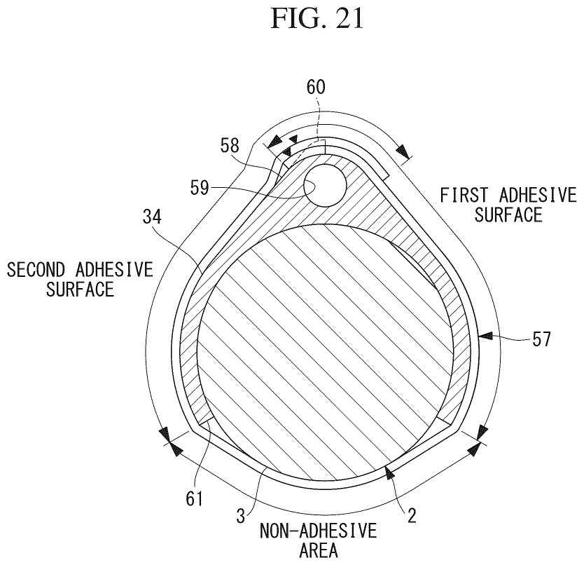

In order to fix the cap 5 to the insertion portion 3 of the endoscope 2 by using the fixture 57, as shown in FIG. 21, in a state in which the distal end of the insertion portion 3 of the endoscope 2 is inserted into the hood part 34, the fixture 57 from which the release paper 65 is peeled off is wrapped around the hood part 34 and the outer circumferential surface of the insertion portion 3. Then, the fixture 57 is tightened until the colored line 57b, at the other end, is disposed at a position beyond the colored line 57a, at the one end, in the circumferential direction of the hood part 34, and the second adhesive surface 63 is applied so as to overlap the front surface at the one end of the fixture 57.

Accordingly, the hood part 34 is elastically deformed, the notch 61 is narrowed, and the insertion portion 3 is tightened by the hood part 34, whereby the cap 5 and the insertion portion 3 are firmly fixed to each other.

Because the non-adhesive area 64 is provided at the position corresponding to the outer circumferential surface of the insertion portion 3, and an adhesive agent is not applied to the outer circumferential surface of the insertion portion 3, it is possible to prevent a situation in which an adhesive agent remains on the outer circumferential surface of the insertion portion 3 after the fixture 57 is removed.

As shown in FIG. 16, the hand operation portion 52 includes a grip part 67 that is gripped by an operator with one hand (for example, the left hand), and a handle part 68 that is operated with the other hand (for example, the right hand). The grip part 67 includes: a columnar body section 70 that has, on an outer surface thereof, at least one recessed section 69 on which, when gripped by the one hand, the index finger or the middle finger of the gripping hand is placed; and a lock button 71 that is located at such a position as to be operatable with a finger (for example, the thumb) of the gripping hand.

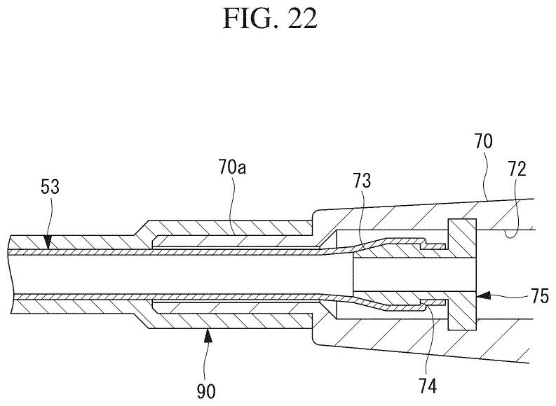

As shown in FIG. 22, the body section 70 has a hollow section 72 through which the coil tube 55 and the wire 56 are inserted in the axial direction. One end of the channel tube 53 inserted from a distal end of the body section 70 is fixed inside the hollow section 72. A tapered distal-end section 73 of a locking member 75, the locking member 75 including the distal-end section 73 and a step section 74 disposed closer to the proximal end than the distal-end section 73 is, is thermally pressed into one end of the channel tube 53, whereby the channel tube 53 is hooked on the step section 74, and the locking member 75 is fixed to the one end of the channel tube 53. The locking member 75 fixed to the channel tube 53 is fixed inside the body section 70, whereby the channel tube 53 can be easily fixed so as not to come off from the body section 70.

The body section 70 has a boss 70a at the distal end thereof. A reinforcing tube (heat shrink tube) 90 covers the boss 70a and the channel tube 53 such that the channel tube 53 is not broken at a distal end of the boss 70a. Because the reinforcing tube 90 is fixed to at least sections of the boss 70a and the channel tube 53, a positional shift does not occur with respect to the channel tube 53 and the boss 70a. A distal-end section of the reinforcing tube 90 is located on the channel tube 53, and a proximal-end section of the reinforcing tube 90 is located on the boss 70a. An outer diameter of the distal-end section of the reinforcing tube 90 is smaller than an outer diameter of the proximal-end section of the reinforcing tube 90.

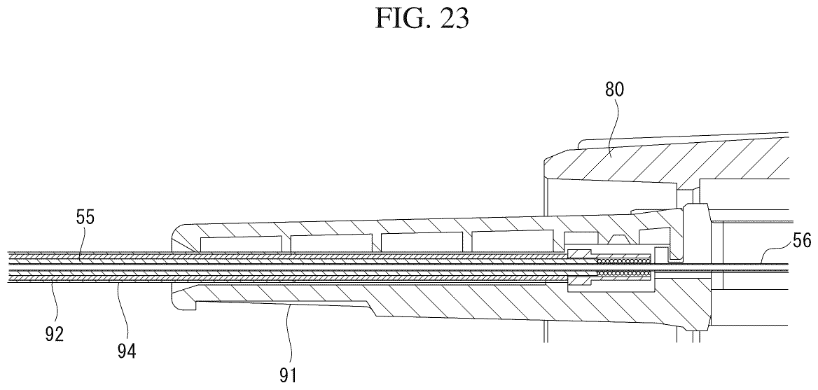

The coil tube 55 protruding from the channel tube 53 and the locking member 75 extends inside the body section 70 (the hollow section 72) in the longitudinal-axis direction. Furthermore, the coil tube 55 is extended from the proximal end of the body section 70, and a proximal end of the coil tube 55 is coupled to a connector 91 of the handle part 68, as shown in FIG. 23.

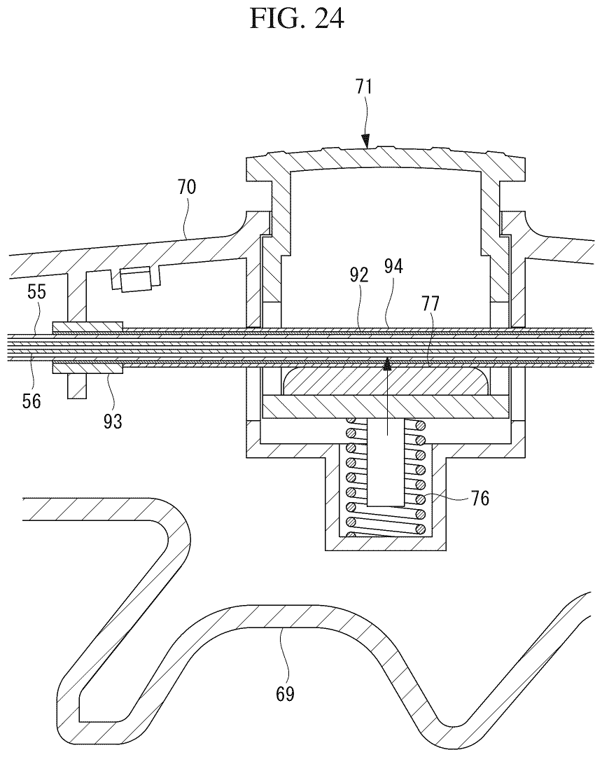

As shown in FIG. 24, the coil tube 55 passes inside a metal pipe 92, so that the coil tube 55 is prevented from widely meandering. It is preferred that the material of the metal pipe be SUS. A distal end of the metal pipe 92 is fixed to the coil tube 55 inside the body section 70 (the hollow section 72). It is preferred that the fixing method be fixing by caulking with a metal part (fixing member) 93, such as brass, for example. A proximal end of the metal pipe 92 is bonded to the coil tube 55 by an adhesive agent or the like inside the connector 91. Because the metal pipe 92 extends between the body section 70 and the connector 91, the coil tube 55 can be maintained in a straight state between the body section 70 and the connector 91 due to the rigidity of the metal pipe 92.

As shown in FIGS. 23 and 24, the outer circumference of the metal pipe 92 is covered with a covering tube (heat shrink tube) 94, as an insulation measure. A distal end of the covering tube 94 is located inside the body section 70 (the hollow section 72), a proximal end of the covering tube 94 is located inside the connector 91, and at least a section of the covering tube 94 is fixed to the metal pipe 92. The metal pipe 92 and the covering tube 94, which covers the metal pipe 92, extend between the body section 70 and the connector 91.

As shown in FIG. 24, the lock button 71 is always biased, by a spring 76, in such a direction as to protrude from the body section 70. A pressing member 77 is provided in the lock button 71, the pressing member 77 being in contact with a side surface of the covering tube 94, which covers the metal pipe 92, when the lock button 71 is not pressed, and being separated from the side surface of the covering tube 94 when the lock button 71 is pressed. In a state in which the lock button 71 is made to protrude from the body section 70, the pressing member 77 is in contact with the covering tube 94. Accordingly, the friction between the pressing member 77 and the covering tube 94 restricts movement of the covering tube 94 in the longitudinal direction with respect to the body section 70. Specifically, the three-layer structure of the covering tube 94, the metal pipe 92, and the coil tube 55 is not advanced or retracted with respect to the body section 70.

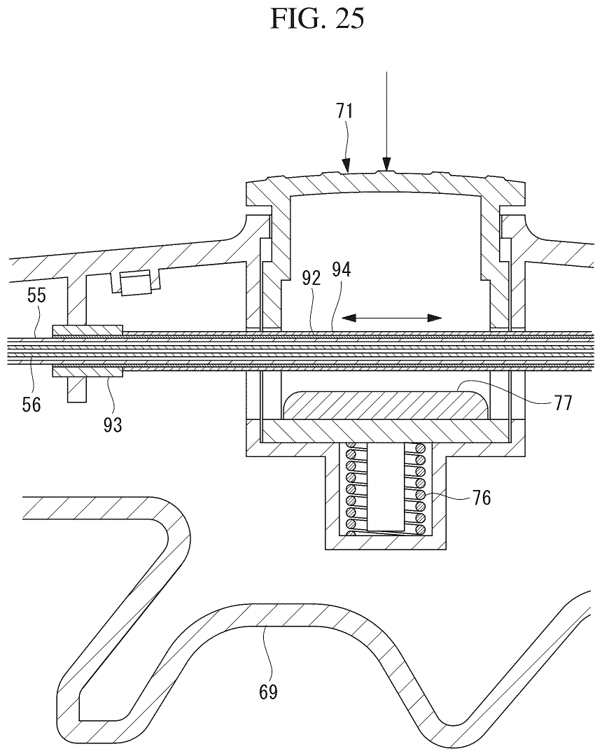

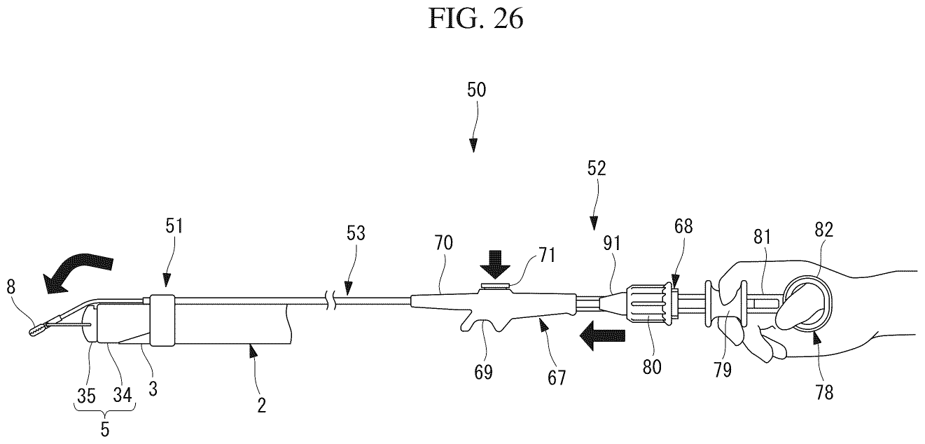

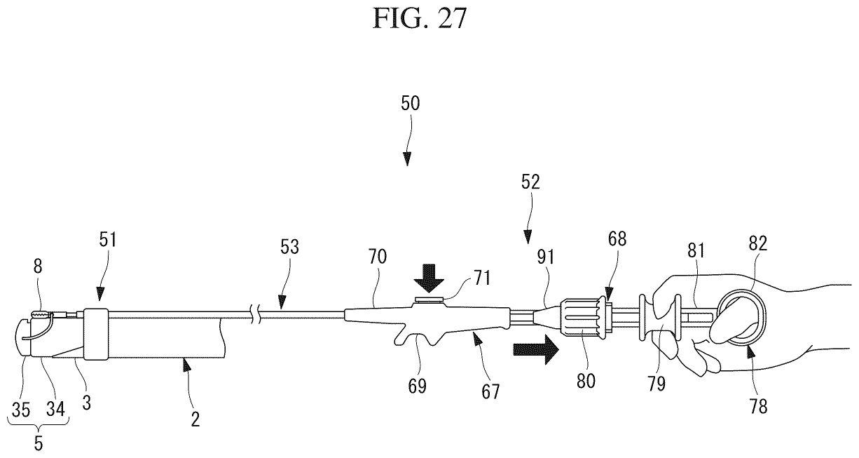

As shown in FIG. 25, when the operator presses the lock button 71, the pressing member 77 is separated from the side surface of the covering tube 94, thereby allowing movement of the three-layer structure of the covering tube 94, the metal pipe 92, and the coil tube 55 with respect to the body section 70. As shown in FIGS. 26 and 27, while pressing the lock button 71, the operator advances or retracts the handle part 68 with respect to the grip part 67, thereby making it possible to move the coil tube 55 in the longitudinal direction to advance or retract the grasping part 8, which is disposed in the distal-end portion 51.

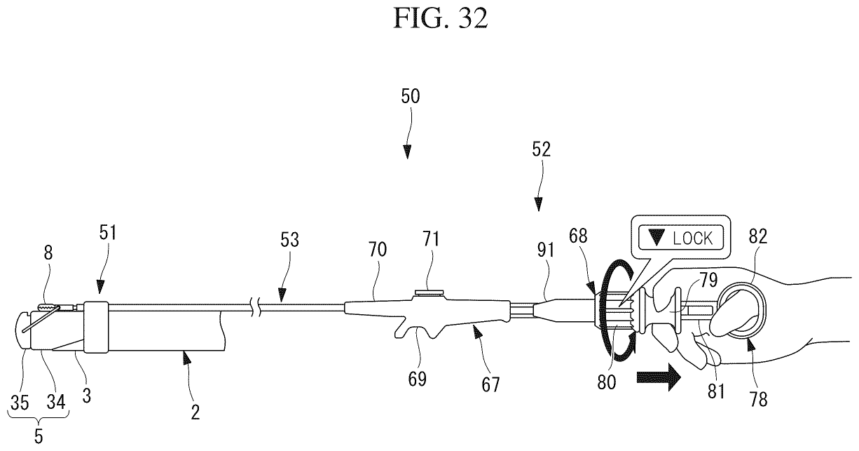

Note that, as shown in FIGS. 26 and 32, the coupling member 6 is tensioned in states in which the grasping part 8 is most advanced and is most retracted, whereas, as shown in FIG. 27, the coupling member 6 becomes loose in a state of transition between these two states.

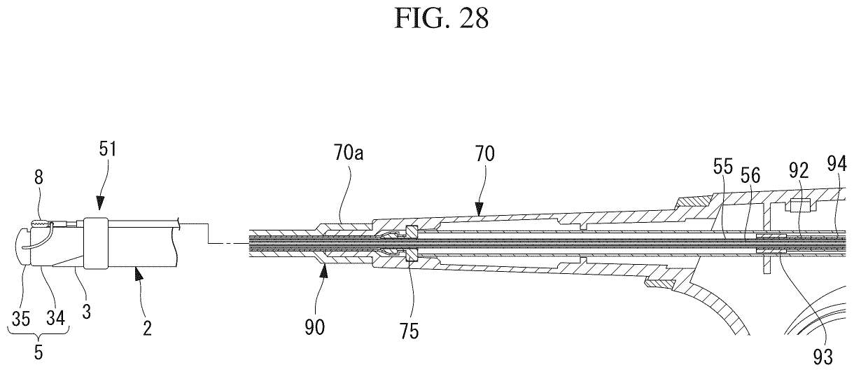

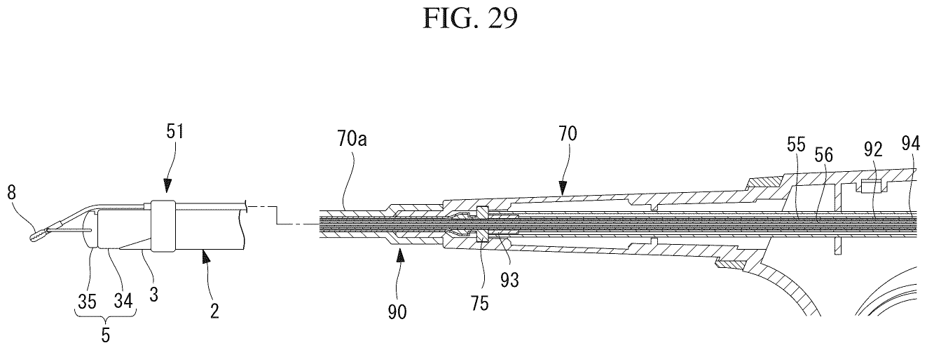

As shown in FIGS. 28 and 29, in a state in which the body part 7 abuts against the protruding section 58, the grasping part 8 is placed in the most-retracted state, and the metal part 93 is located at a position away from the locking member 75. When the metal part 93 abuts against the locking member 75, the grasping part 8 is placed in the most-advanced state.

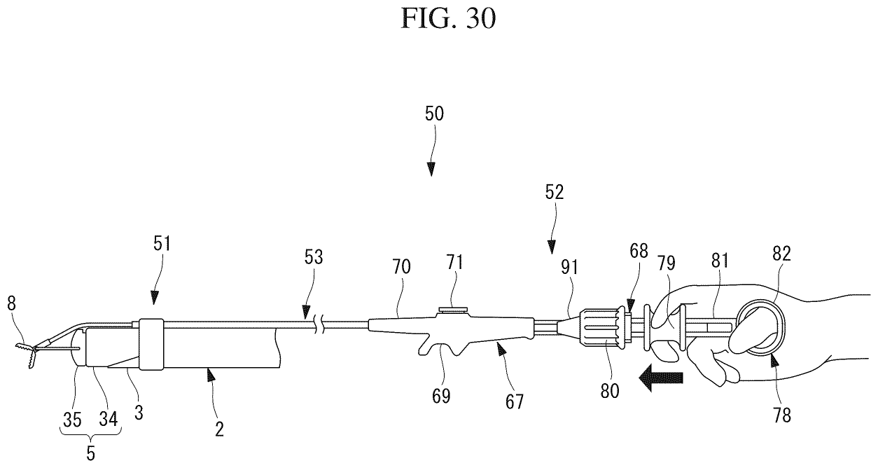

As shown in FIG. 30, the handle part 68 includes: a support part 78 that is supported by the operator with the thumb of the right hand; a slider 79 that is operated with the index finger and the middle finger of the right hand; a jaw lock 80 that locks the grasping part 8 in a closed state; and the connector 91, which supports the jaw lock 80 so as to be rotatable about the longitudinal axis. The support part 78 includes a guide rail 81 that extends in the front-back direction, and a ring part 82 that is provided at a proximal end of the guide rail 81 and into which the operator inserts the thumb of the right hand.

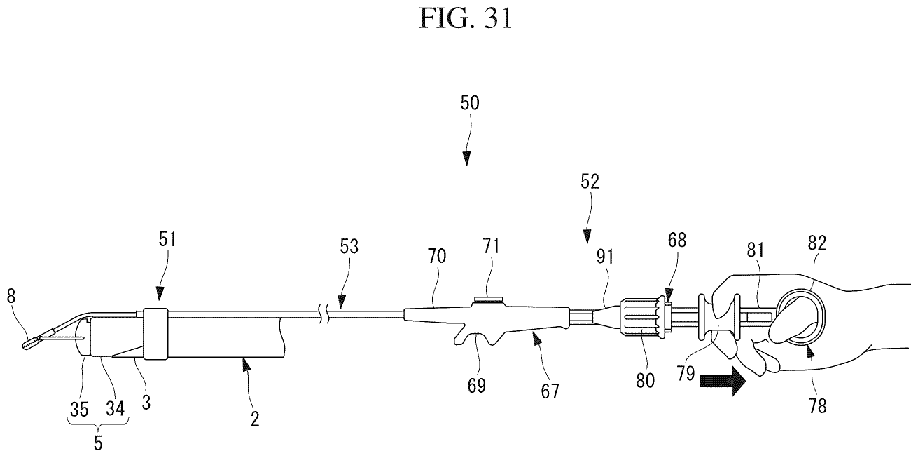

A proximal end of the wire 56, a distal end of which is connected to the grasping part 8, is fixed to the slider 79. In order to open and close the grasping part 8, which is disposed in the distal-end portion 51, as shown in FIGS. 30 and 31, the index finger and the middle finger of the right hand are hooked on the slider 79 with the thumb of the right hand being inserted into the ring part 82, and the slider 79 is made to slide in the front-back direction along the guide rail 81. Accordingly, the wire 56 in the coil tube 55 is advanced and retracted in the longitudinal direction, thereby making it possible to open and close the grasping part 8.

As shown in FIG. 32, the jaw lock 80 can be moved along the guide rail 81, thereby being brought into contact with the slider 79, and can be rotated about the front-back axis at that position, thereby being fixed to the guide rail 81. Accordingly, for example, in a state in which tissue is grasped by the grasping part 8, movement of the slider 79 can be restricted by the jaw lock 80, thus making it possible to maintain the state in which the tissue is grasped by the grasping part 8.

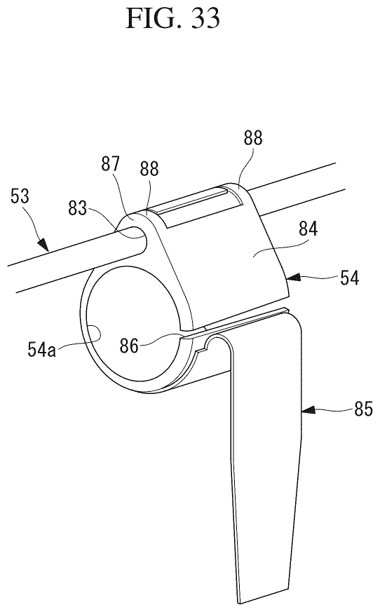

As shown in FIG. 33, the intermediate fixing part 54 includes: an inner hole 54a into which the insertion portion 3 of the endoscope 2 can be inserted; a cylindrical body section 84 that has a through-hole 83 into which the channel tube 53 can be inserted; and a fixture 85 that is wrapped around the body section 84 in the circumferential direction. The body section 84 has, at a section thereof in the circumferential direction, a slit 86 that is obtained by cutting a wall thereof along the axial direction. Furthermore, the body section 84 is formed of an elastic material, and the width of the slit 86 can be increased or decreased through elastic deformation.

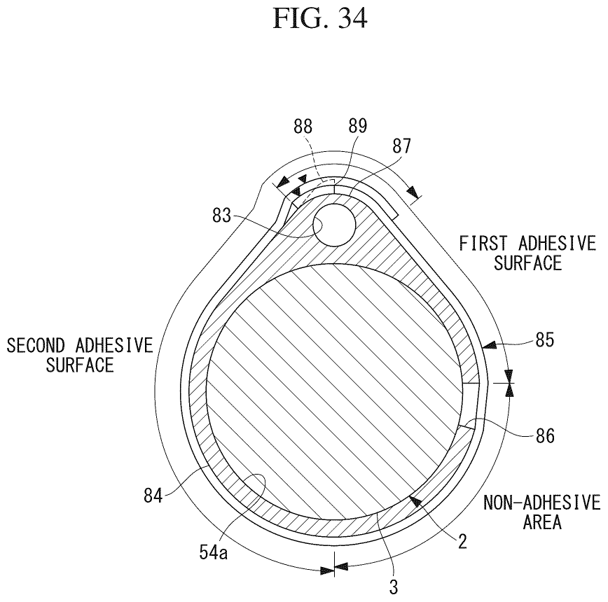

The length of the inner hole 54a of the body section 84 in the circumferential direction is set so as to be less than the length of the outer circumferential surface of the insertion portion 3 of the endoscope 2 in the circumferential direction. Accordingly, as shown in FIG. 34, when the insertion portion 3 of the endoscope 2 is disposed in a state of being inserted into the inner hole 54a, an inner surface of the inner hole 54a is brought into close contact with the outer circumferential surface of the insertion portion 3, whereby the slit 86 is in an open state.

A protruding section 87 that protrudes radially outward is provided, on the body section 84, at a position different from that of the slit 86 in the circumferential direction, and the through-hole 83 is provided in the protruding section 87 so as to be parallel to the inner hole 54a. A pair of protrusions 88 that are used to position the fixture 85 in the axial direction are provided at the apex of the protruding section 87.

The fixture 85 has the same structure as the fixture 57, shown in FIG. 19, which is used to fix the cap 5 to the insertion portion 3. Specifically, the fixture 85 is a tape that has the adhesive surfaces 62 and 63 and the non-adhesive area 64 on the back surface and has the colored lines 57a and 57b formed on the front surface. At one end of the fixture 85, a step section 89 is provided to position, in the circumferential direction of the body section 84, the fixture 85 disposed between the protrusions 88 of the protruding section 87 and thus positioned in the axial direction with respect to the body section 84. The step section 89 is aligned with the protrusions 88 of the protruding section 87, thereby making it possible to position the fixture 85 in the circumferential direction of the body section 84. The non-adhesive area 64 is provided at a position corresponding to the slit 86 of the body section 84 when the fixture 85 is wrapped around the body section 84.

The fixture 57 of the cap 5 and the fixture 85 of each of the intermediate fixing parts 54 are each shipped with the first adhesive surface 62 being adhered to the outer circumferential surface of the cap 5 or the body section 84 and with the release paper 65 being applied to the second adhesive surface 63. In order to mount the endoscope treatment tool 1, 50 on the insertion portion 3 of the endoscope 2, the distal end of the insertion portion 3 of the endoscope 2 is inserted into the hood part 34 of the cap 5, and an intermediate position of the insertion portion 3 is accommodated inside the inner hole 54a of the body section 84 by widening the slit 86 of the body section 84 of the intermediate fixing part 54. In this state, the release paper 65 on the second adhesive surface 63 is peeled off, and the second adhesive surface 63 is applied while the fixture 57, 85 is pulled and is wrapped around the outer circumferential surface of the hood part 34 or the body section 84, whereby the endoscope treatment tool 1, 50 is mounted on the insertion portion 3 of the endoscope 2.

The above-described embodiments can also lead to the following aspects.

A first aspect of the present invention provides an endoscope treatment tool including: a cylindrical cap that is attached to an outer side of a distal-end section of an endoscope, the cylindrical cap having a first radial direction and a second radial direction intersecting each other; a treatment tool that has, at a distal end, a grasping part for grasping living tissue, the treatment tool being disposed, at an outer side of the cap in the first radial direction, along a longitudinal direction of the cap, the treatment tool being supported by the cap, at a position closer to a proximal end than the grasping part is, so as to be movable in the longitudinal direction; and a coupling member that couples the treatment tool with the cap, the coupling member having a linear shape, wherein the coupling member has a swing section that is disposed outside the cap and that is supported by the cap so as to be swingable about a swing axis extending in the second radial direction; and the swing section couples the cap with the treatment tool so as to allow relative movement in the second radial direction, and, in response to pressed forward in the longitudinal direction by the treatment tool moved forward in the longitudinal direction, swings about the swing axis while generating a tensile force and bending the treatment tool inward in the first radial direction.

According to this aspect, when the cap is attached to the distal-end section of the endoscope such that the first radial direction of the cap and the up-down direction of the endoscope become substantially parallel to each other and such that the treatment tool is disposed at an upper side of the endoscope, the treatment tool is disposed outside the endoscope along the longitudinal direction of the endoscope. Then, when the treatment tool is moved forward in the longitudinal direction with respect to the cap and the endoscope, the swing section of the coupling member, which couples the treatment tool with the cap, swings about the swing axis, which intersects the up-down direction of the endoscope, toward the lower side of the endoscope, while bending the treatment tool downward. Accordingly, the grasping part moves from the upper side of the endoscope toward the lower side thereof. Therefore, merely by pushing the treatment tool forward with respect to the cap and the endoscope, it is possible to bring the grasping part close to a target section of the living tissue located in a lower area in the field of view of the endoscope and to grasp the target section. Furthermore, merely by pulling the treatment tool rearward with respect to the cap and the endoscope, the target section grasped by the grasping part can be raised.

In this case, because the treatment tool and the cap are coupled via the coupling member in a manner allowing relative movement in the second radial direction, which intersects the up-down direction of the endoscope, i.e., in the left-right direction of the endoscope, the treatment tool and the endoscope can be moved independently of each other. Specifically, when the distal end of the endoscope is moved in the left-right direction through an operation of the bending section, the grasping part grasping the target section stays stationary without following the movement of the endoscope. Accordingly, while the target section, which is raised by the grasping part, is made to stay stationary, treatment, such as cutting, can be easily performed on the target section by operating the endoscope.

In the above-described first aspect, the treatment tool may have a hole that is provided at a position closer to the proximal end than the grasping part is and that penetrates in a direction intersecting a longitudinal direction of the treatment tool; the swing section may be inserted through the hole so as to be movable in a longitudinal direction of the swing section; and both end sections of the coupling member that sandwich the swing section in a longitudinal direction thereof may be supported by the cap at two places opposed to each other in the second radial direction, so as to be swingable about the swing axis.

By doing so, relative movement of the treatment tool and the cap in the second radial direction can be realized through movement of the swing section inside the hole in the treatment tool. Furthermore, the swing section can swing about the swing axis passing through the two places on the cap, at which both end sections of the swing section are supported.

In the above-described first aspect, the cap may have two holes that penetrate in a radial direction of the cap, at two places opposed to each other in the second radial direction; an intermediate position of the swing section in a longitudinal direction thereof may be fixed to the treatment tool; and both end sections of the coupling member that sandwich the swing section in a longitudinal direction thereof may be inserted through the two holes so as to be movable in a longitudinal direction of the coupling member.

By doing so, relative movement of the treatment tool and the cap in the second radial direction can be realized through movement of the coupling member inside the holes in the cap. Furthermore, the swing section can swing about the swing axis passing through the two holes in the cap, into which both end sections of the swing section are inserted.

In the above-described first aspect, the swing section of the coupling member may have a fixed length.

By doing so, the movement track of the grasping part through swing of the swing section can be fixed.

In the above-described first aspect, the swing section of the coupling member may be switched from a tensed state in which the tensile force is generated to a relaxed state.

By doing so, after the target section is grasped by the grasping part, the swing section is switched from the tensed state to the relaxed state, and the treatment tool is pushed forward in the longitudinal direction while the target section is grasped by the grasping part, thereby making it possible to raise the target section also in a direction away from the distal end of the endoscope in the longitudinal direction.

In the above-described first aspect, the cap may support the treatment tool at a proximal-end section of the cap.

By doing so, it is possible to secure a long length of a section that is bent when the treatment tool is pushed forward in the longitudinal direction.

A second aspect of the present invention provides an endoscope system including: an endoscope; and one of the above-described endoscope treatment tools.

According to the present invention, an advantageous effect is afforded in that a treatment tool guided into the field of view of an endoscope via the outside of the endoscope is operated independently of the movement of the endoscope, thereby making it possible to easily perform treatment.

### REFERENCE SIGNS LIST

1 endoscope treatment tool
2 endoscope
3 insertion portion
3a distal-end rigid section
3b bending section
4 treatment tool
4a hole
5 cap
5a, 5b hole
6 coupling member
6a swing section
7 body part
8 grasping part (grasper)
8a, 8b grasping piece (grasper)
9 support part
9a channel
10 protrusion
20 treatment tool
A swing axis
X affected tissue (living tissue)

What is claimed is:

1. A endoscopic tool comprising:
a cap configured to be attached to a distal end of a flexible tubular member, the cap having a channel extending longitudinally to accept the distal end of the flexible tubular member;
a guide hole having an opening on an outer peripheral surface of the cap; and
a filament extending through the guide hole such that the filament and the outer peripheral surface of the cap contact each other.

2. The endoscopic tool of claim 1, further comprising a grasper connected to the filament at a distal end portion of the grasper.

3. The endoscopic tool of claim 2, wherein
the grasper is disposed on a distal end of a sheath extending along an outside of the flexible tubular member; and
a handle is mounted on a proximal end of the sheath, a proximal end of the filament is spaced longitudinally from the handle.

4. The endoscopic tool of claim 1, wherein the filament extends through the guide hole without an additional element around the guide hole.

5. The endoscopic tool of claim 1, wherein a proximal end of the filament outside the patient is configured to be withdrawn proximally by an operator.

6. The endoscopic tool of claim 1, wherein the flexible elongate tubular member is an endoscope.

7. The endoscopic tool of claim 2, wherein the grasper and the filament are movable longitudinally by pulling a proximal end of the filament extended outside the patient.

8. The endoscopic tool of claim 1, wherein the filament contacts the cap in a state in which the filament is withdrawn proximally.

9. A endoscopic tool comprising:
a cap configured to be attached to a distal end of a flexible tubular member, the cap having a channel extending longitudinally to accept the distal end of the flexible tubular member;
a support portion disposed on the outside of the cap; and
a filament configured to swing around the support portion.

10. The endoscopic tool of claim 9, wherein
the cap further having a guide hole having an opening on an outer peripheral surface of the cap, the filament extending through the guide hole; and
the support portion is arranged between the guide hole and a grasper attached to the filament.

11. The endoscopic tool of claim 9, wherein the support portion protrudes from an outer surface of the cap.

12. The endoscopic tool of claim 9, further comprising a grasper connected to the filament at a distal end portion of the grasper.

13. The endoscopic tool of claim 12, wherein
the grasper is mounted on a distal end of a sheath extending along an outside of the flexible tubular member; and
a handle is mounted on a proximal end of the sheath, a proximal end of the filament is spaced longitudinally from the handle.

14. The endoscopic tool of claim 9, wherein the filament extends through a guide hole in the cap without an additional element around the guide hole.

15. The endoscopic tool of claim 9, wherein a proximal end of the filament outside the patient is configured to be withdrawn proximally by an operator.

16. The endoscopic tool of claim 9, wherein the flexible elongate tubular member is an endoscope.

17. The endoscopic tool of claim 9, wherein the grasper and the filament are movable longitudinally by pulling a proximal end of the filament extended outside the patient.

18. A system comprising:
a flexible tubular member;
a cap configured to be attached to a distal end of the flexible tubular member, the cap having a channel extending longitudinally to accept the distal end of the flexible tubular member;
a guide hole having an opening on an outer peripheral surface of the cap; and
a filament extending through the guide hole such that the filament and the outer peripheral surface of the cap contact each other.

19. The system of claim 18, wherein the filament extends through the guide hole without an additional element around the guide hole.

20. The system of claim 18, wherein a proximal end of the filament outside the patient is configured to be withdrawn proximally by an operator.

21. The system of claim 18, wherein the flexible elongate tubular member is an endoscope.

22. The system of claim 18, wherein the filament and the grasper are movable longitudinally by pulling a proximal end of the filament extended outside the patient.
